(19) United States
(12) Patent Application Publication
(10) Pub. No.: US 2026/0062863 A1
(43) Pub. Date: Mar. 5, 2026

(54) PHOTOCLEAVAGE OF INDICAN

(71) Applicant: DANMARKS TEKNISKE UNIVERSITET, Kongens Lyngby (DK)

(72) Inventors: Ditte Hededam WELNER, Kongens Lyngby (DK); Gonzalo Nahuel Bidard COSTOYA, Kongens Lyngby (DK); David TEZE, Kongens Lyngby (DK); Natalia PUTKARADZE, Kongens Lyngby (DK); Folmer FREDSLUND, Kongens Lyngby (DK); Charlotte ULDAHL JANSEN, Kongens Lyngby (DK); Katrine QVORTRUP, Kongens Lyngby (DK)

(21) Appl. No.: 19/108,219

(22) PCT Filed: Sep. 8, 2023
(86) PCT No.: PCT/EP2023/074715

§ 371 Date: Mar. 3, 2025

(30) Foreign Application Priority Data
Sep. 9, 2022 (EP) 22194865.6

(51) Int. Cl. D06P 5/20; C09B 7/00; C09B 67/44; C12P 19/60; D06P 1/22; D06P 3/60

(52) U.S. Cl. CPC D06P 5/2005; C09B 7/00; C09B 67/0083; C12P 19/60; D06P 1/228; D06P 3/6025

(57) ABSTRACT

The present invention concerns the use of light to convert indicant to indigo. The invention is particularly useful in dyeing textiles from indican by simple light exposure, thereby providing a greener alternative to current industrial processes for colored fabrics and other products.

### FIELD OF THE INVENTION

The present invention concerns the use of light to dye textiles from indican, thereby providing a greener alternative to current industrial processes for colored fabrics and other products.

### BACKGROUND OF THE INVENTION

Blue denim is traditionally dyed with chemically synthesized indigo under harsh environmentally challenging conditions. As a final step in the synthesis, indigo forms spontaneously from indoxyl by oxidation by air, but for use in dyeing, indigo further needs to be solubilized with a strong reducing agent (e.g. Na₂S₂O₄), which is likewise environmentally challenging (FIG. 1).

Glycosyltransferase (GT) enzymes can be applied as a green biotech alternative to current industrial processes for blue denim production (FIG. 1). GTs glycosylate many different chemicals, including indoxyl (the indigo dye precursor). Specifically, the hydroxyl group of chemically synthesized indoxyl may be glycosylated by glycosyltransferase, thereby protecting the reactive functional group and generating the stable soluble (colorless) indican molecule. Indican may then later be hydrolyzed by beta-glucosidase (BGL) back to indoxyl which can then spontaneously oxidize to form blue indigo directly on the fabric (FIG. 1). This is a “green” alternative to the traditional industrial process, providing an alternative solution to the final steps of the indigo dyeing process, whereby the use of the harsh strong reducing agent is avoided.

The present invention offers further improvement over the art in providing a simpler, green and cheaper process for indigo dyeing.

### SUMMARY OF THE INVENTION

The present invention concerns a method for producing indigo, comprising the steps of
a. providing an aqueous solution comprising indican,
b. exposing the indican to a light source,
   wherein the indican is exposed to sufficient radiant energy by the light source to convert at least 50% of the indican to indigo; and wherein the glycosidic bond in indican is cleaved solely by said light exposure.

The present invention further concerns a method of dyeing a product, such as a yarn, textile, or fabric, with indigo, said method comprising the steps of
a. providing indican in an aqueous solution,
b. applying the indican solution from step (a) to said product, and
c. exposing the indican-treated product from step (b) to a light source, to obtain a dyed product,
   wherein the glycosidic bond in the indican is cleaved solely by said light exposure.

In preferred embodiments, the aqueous solution in step (a) of the methods of the invention is acidic.

In preferred embodiments, the wavelength of the light source used for cleaving the glycosidic bond in indican is 300-470 nm.

In preferred embodiments, the method of dyeing a product comprises a pre-step of glycosyltransferase treatment for glycosylating indoxyl to form the indican.

The invention further concerns the use of light for conversion of indican to indigo.

### DESCRIPTION OF THE INVENTION

### Brief Description of the Figures

FIG. 1: The ‘traditional process’ involves chemically synthesized indigo and addition of reducing agents (e.g. sodium dithionite, Na₂S₂O₄) to the indigo vat for reduction to dye-competent, soluble leucoindigo. In the ‘2× enzymatic process’, indoxyl is glucosylated at the C3 hydroxyl group giving indican as product. The glucoside (indican) is stable and can be stored. The glucosyl group is removed on fabric, during the dyeing step, allowing the regenerated indoxyl to oxidize to indigo on the fabric. No reducing agent is required when dyeing with indican. In the ‘enzymatic-light process’, indoxyl is glycosylated to form indican, and the indican is on the fabric exposed to light, whereby indigo is formed. GT=glycosyltransferase, BGL=β-glycosyl hydrolase.

FIG. 2: Illustration of start, intermediate, and end product structures in the conversion of indican to indigo by the ‘2× enzymatic process’ (top) and the ‘enzymatic-light process’ (bottom).

FIG. 3: Picture of cotton fabric patches that have been dyed using indican (top), and their CIELAB values (bottom) for characterization of the indigo color. On the left: Indican was hydrolyzed by BGL at pH 9. On the right: Indican was photocleaved by LED.

FIG. 4: Picture of photocleavage of indican in solution using (A) Mercury UV Bulb, (B) Household Bulb, and (C) Artificial Sunlight Bulb as light sources; and where different solvents were tested as specified on the picture.

FIG. 5: UV chromatograms of products of indican photocleavage at different HCl (aq) concentrations. The annotated peak was confirmed to be indigo by NMR.

FIG. 6: Picture of cotton fabric swatches that have been dyed once (left hand side) or twice (right hand side) using photocleavage of indican, and their respective CIELAB values for characterization of the indigo color, demonstrating that repeated dipping allows for dynamic coloring analogous to the conventional process.

FIG. 7: Picture of cotton fabric swatches that have been dyed using different mixing modes, and their respective CIELAB values for characterization of the indigo color.

FIG. 8: Picture of cotton fabric swatches that have been dyed at different exposure times and with different distances between the lamp and fabric, and their respective CIELAB values for characterization of the indigo color.

FIG. 9: Indigo formation from indican by LEDs with different wavelengths.

FIG. 10: Picture of cotton fabric swatches treated at reaction conditions as specified in Table 10.

FIG. 11: Dyeing additional fabric materials. (A) STEELOUT sample (90/10 wool/nylon), (B) DIVINA (100% wool).

FIG. 12: Examples of yarn coloring (in duplets). Conditions: indican solution is 7 mg/mL in milliQ water, LED light exposure 3 hrs., distance between light and sample 7 cm, (A) 3 hrs. soak, (B) 6 hrs. soak, (C) sample (A) repeated twice.

FIG. 13: Formation of indican by glycosyl transferase PtUGT1 mut87.

### ABBREVIATIONS, TERMS, AND DEFINITIONS

‘Photocleavage’ is the cleavage of a chemical bond by photolysis—i.e. a chemical reaction in which a compound is decomposed after absorbing a photon.

‘Light’ is in the present application not limited to visible light, but may also cover a broader range of wavelengths, such as both the visible spectrum as well as ultraviolet (UV) and infrared (IR).

‘Radiant flux’ or ‘radiant power’ is the radiant energy (light) emitted, reflected, transmitted, or received per unit time. The SI unit of radiant flux is the watt (W), one joule per second (J/s).

‘Irradiance’ is the radiant flux received by a surface per unit area. The SI unit of irradiance is the watt per square metre (W/m²). Irradiance may also be called intensity.

‘Radiant exposure’ is the radiant energy (light) received by a surface per unit area, or equivalently the irradiance of a surface, integrated over time of irradiation. The SI unit of radiant exposure is the joule per square metre (J/m²).‘LED’ is a light-emitting diode. ‘LED lamp’ or ‘LED light bulp’ is an electric light that produces light using LEDs. LEDs offer efficient lighting in a range of desired wavelengths.

‘OLED’ is an organic light-emitting diode, also known as organic electroluminescent (organic EL) diode, in which the emissive electroluminescent layer is a film of organic compound that emits light in response to an electric current.

‘Dye precursor’ is herein defined as a compound that can give rise to dyed material upon one or more chemical transformations.

Glycosyl transferase (GT) are enzymes that catalyze the formation of the glycosidic linkage.

‘Glycosidic bond’ or ‘glycosidic linkage’ is a covalent bond that joins a carbohydrate (sugar) molecule to another group. In the present invention, indican comprises such glycosidic bond.

‘Nucleotide sugar’ is herein defined as a molecule in which a sugar is bound to a nucleotide via a glycosidic bond; wherein the sugar is a monosaccharide, such as glucose, rhamnose, xylose, arabinose. Nucleotide sugars act as glycosyl donors in glycosylation reactions; those reactions are catalyzed by glycosyltransferases.

‘Amino acid sequence identity’: The term “sequence identity” as used herein, indicates a quantitative measure of the degree of similarity between two amino acid sequences of essentially equal length. The two sequences to be compared must be aligned to give a best possible fit, by means of the insertion of gaps or alternatively, truncation at the ends of the protein sequences. The sequence identity can be calculated as ((Nref-Ndif)100)/(Nref), wherein Ndif is the total number of non-identical residues in the two sequences when aligned and wherein Nref is the number of residues in one of the sequences. Sequence identity calculations are preferably automated using the BLAST program e.g. the BLASTP program (Pearson W. R and D. J. Lipman (1988)) (www.ncbi.nlm.nih.gov/cgi-bin/BLAST). Sequence alignment may be performed using program MAFFT24 (Multiple Alignment using Fast Fourier Transform; Katoh et al 2019) using default parameters (SCORING MATRIX: blosum62, gap opening penalty: 1.53, gap extension penalty 0.123).

Preferably, the numbers of substitutions, insertions, additions or deletions of one or more amino acid residues in the polypeptide as compared to its comparator polypeptide is limited, i.e. no more than 1, 2, 3, 4, 5, 6, 7, 8, 9, or 10 substitutions, no more than 1, 2, 3, 4, 5, 6, 7, 8, 9, or 10 insertions, no more than 1, 2, 3, 4, 5, 6, 7, 8, 9, or 10 additions, and no more than 1, 2, 3, 4, 5, 6, 7, 8, 9, or 10 deletions. Preferably the substitutions are conservative amino acid substitutions: limited to exchanges within members of group 1: Glycine, Alanine, Valine, Leucine, Isoleucine; group 2: Serine, Cysteine, Selenocysteine, Threonine, Methionine; group 3: Proline; group 4: Phenylalanine, Tyrosine, Tryptophan; Group 5: Aspartate, Glutamate, Asparagine, Glutamine; Group 6: Histidine. Lysine, Arginine.

### DETAILED DESCRIPTION OF THE INVENTION

The present invention concerns the use of light to convert indican to indigo, which is particularly useful in dyeing textiles such as demin. Indican is a glycoside of the indigo precursor indoxyl. Indoxyl compounds may under aerobic conditions spontaneously dimerize and form colored compounds, which may be used as dyes, such as for dyeing fabrics or other products. The dimerized colored compounds are insoluble in an aqueous solution. Glycosylation of indoxyl stabilizes the compound, prevents dimerization, and thereby provides the soluble dye precursor indican.

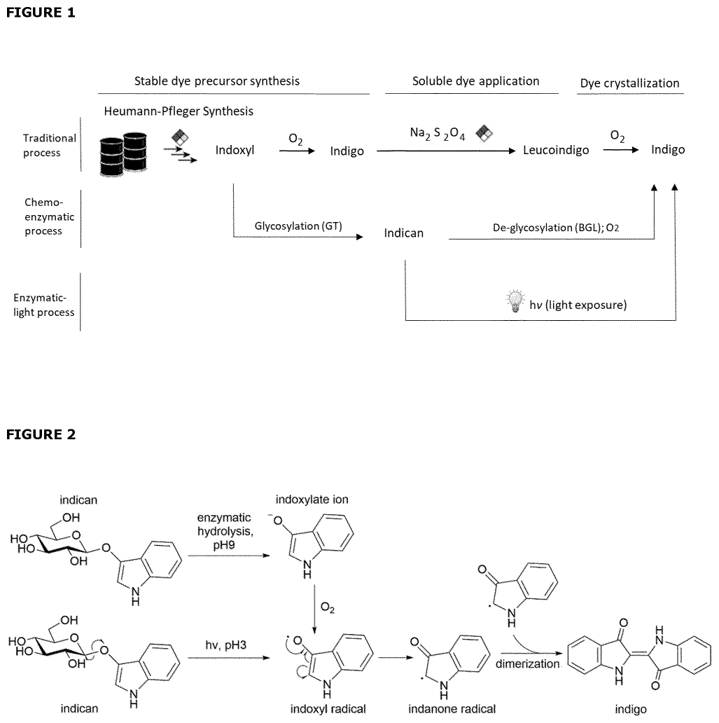

Indican has previously been used to dye denim using enzymatic hydrolysis to break the glycoside bond, followed by the spontaneous reaction under aerobic conditions leading to indigo formation (FIG. 1). The present invention provides a method of using light instead of an enzyme or chemical, thereby making the process both simpler, green and cheaper and more accessible than current known technologies (FIG. 1).

No matter the route—traditional chemical route, 2× enzymatic route, or enzymatic-light route—the final compound giving rise to the blue denim color is the same chemical entity, namely indigo (FIG. 1).

### I. Conversion of Indican to Indigo

The present inventors have discovered that indican can be converted to indigo by simple light exposure. The present invention provides a method for converting indican to indigo by light exposure. Without wishing to be bound by theory, it is speculated that the light source converts the indican (in solution) to indoxyl radical by photocleavage, and that the indoxyl radical further spontaneously dimerizes to form indigo (FIG. 2).

In one aspect, the invention provides a method for converting indican to indigo, comprising the steps of:
a. providing indican in an aqueous solution,
b. exposing the indican to light,
   wherein the conversion is mediated solely by said light exposure.

In one embodiment, the invention provides a method for converting indican to indigo, comprising the steps of:
a. providing an aqueous solution comprising indican,
b. exposing the indican to a light source,
   wherein the indican is exposed to sufficient radiant energy by the light source to convert at least 20, 30, 40, 50, 60, 70, 80 or even 90% of the indican to indigo; and wherein the glycosidic bond in indican is cleaved solely by said light exposure.

In one preferred embodiment, the invention provides a method for converting indican to indigo, comprising the steps of:
a. providing an aqueous solution comprising indican,
b. exposing the indican to a light source,
   wherein the indican is exposed to sufficient radiant energy by the light source to convert at least 50% of the indican to indigo; and wherein the glycosidic bond in indican is cleaved solely by said light exposure.

In one preferred embodiment, the invention provides a method for converting indican to indigo, comprising the steps of:
a. providing an aqueous solution comprising indican,
b. exposing the indican to a light source,
   wherein the indican is exposed to sufficient radiant energy by the light source to cleanly convert indican to indigo, consuming at least 10, 20, 30, 40, 50, 60, 70, 80, or 90% of the indican; and wherein the glycosidic bond in indican is cleaved solely by said light exposure.

The light exposure mediates the conversion of indican to indigo by cleaving the glycosidic bond in indican. Particularly, in one preferred embodiment, the invention provide a method for conversion of indican to indigo which does not comprise the use of beta-glycosyl hydrolase enzymes (Enzyme classification number E.C 3.2.1.21).

In preferred embodiments, the conversion of indican to indigo comprises formation of an indoxyl radical as an intermediate product.

In preferred embodiments, the conversion of indican to indigo comprises (i) formation of an indoxyl radical as an intermediate product, and (ii) a spontaneous dimerization reaction and oxidation for formation of indigo.

### I.i the Light Source

As evidenced in the examples, the conversion of indican to indigo by light exposure works with a range of different light sources, including natural sunlight, artificial sunlight bulbs, as well as specific LEDs, obtaining the traditional blue denim color sought after by the consumers.

In some embodiments, the light source may comprise one or more lasers with a specific or adjustable wavelength, an LED/OLED, an incandescent lamp, a mercury lamp, a UV lamp, an arc lamp, an argon lamp, or any other gas lamp (e.g. neon and krypton). In some embodiments, the light source may comprise natural sunlight or an artificial sunlight bulb. The light source can be pulsating (i.e., turned on for a finite amount of time in bursts) or continuous, gated by physical shutters, filtered, amplified, dampened, polarized, or otherwise manipulated to generate a light used to photocleave indican.

The glycosidic bond in indican is excited with a specific wavelength or a range of wavelengths to induce photocleavage in an efficient manner. In one embodiment, the light source comprises a wavelength between 300-470 nm, 300-450 nm, between 310-440 nm, between 320-430 nm, between 330-420 nm, between 340-410 nm, between 350-400 nm, between 360-390 nm, between 370-380 nm, such as a wavelength around 375 nm.

In one preferred embodiment, the wavelength of the light source for concerting indican to indigo—i.e. for cleaving the glycosidic bond in indican—is between 300-450 nm, between 310-440 nm, between 320-430 nm, between 330-420 nm, between 340-410 nm, between 350-400 nm, between 360-390 nm, between 370-380 nm.

In one embodiment, the wavelength of the light source is 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, or 500 nm.

### II. a Method for Producing Indigo and a Method of Dyeing a Product

In one aspect, the invention provides a method for producing indigo, comprising the steps of
a. providing an aqueous solution comprising indican,
b. exposing the indican to a light source,
   wherein the indican is exposed to sufficient radiant energy by the light source to convert at least 10, 20, 30, 40, 50, 60, 70, 80, or 90% of the indican to indigo; wherein the glycosidic bond in indican is cleaved solely by said light exposure.

In one further aspect, the present invention provides a method of dyeing a product with indigo, comprising the steps of
a. providing indican in an aqueous solution,
b. applying the indican solution from step (a) to said product, and
c. exposing the indican-treated product from step (b) to a light source to obtain a dyed product,
   wherein the glycosidic bond in indican is cleaved solely by said light exposure.

The method of dyeing a product with indigo may be repeated multiple times—such as two, three, four, five or more times, to obtainer a deeper/darker coloring of the product.

Particularly, the invention provides methods for producing indigo from indican, and optionally using said indigo in dyeing a product, wherein said methods do not comprise the use of a beta-glycosyl hydrolase enzyme (Enzyme classification number E.C 3.2.1), such as beta-glucoside (E.C. 3.2.1.21).

In some embodiments, the product intended for dyeing by the methods disclosed herein, may be selected from yarn, textile, fabrics, and similar products. In a preferred embodiment, the product is a yarn or a textile.

II.i. Providing Indican

Indigo is in the present invention produced from indican. Indican may be provided by different means. In one embodiment, indican may be obtained from indoxyl by an enzymatic glycosylation reaction. Glycosyltransferase enzymes possess glycosyltransferase activity (enzyme classification enzyme classification EC: 2.4.1.-) for glycosylating compounds having a reactive group.

In one embodiment, indican is provided by: providing (i) indoxyl, (ii) a nucleotide sugar, and (iii) a polypeptide having glycosyltransferase enzyme activity, mixing components (i), (ii) and (iii), preferably at reaction conditions wherein less than 2% free oxygen is present, letting the mixture react to obtain indican.

In the present invention, the glycosyltransferase enzyme glycosylates indoxyl to form indican, the soluble stable indigo dye pre-cursor. In a preferred embodiment, the glycosyltransferase enzyme has indoxyl-UDP glucosyltransferase activity (enzyme classification EC: 2.4.1.220). In one embodiment, the amino acid sequences of the polypeptide having glycosyltransferase enzyme activity has at least 75% sequence identity to UDP-dependent glycosyltransferase (PtUGT1, SEQ ID NO. 2) from Polygonum tinctorium/Persicaria tinctoria, such as at least 76, 77, 78, 79, 80, 81, 82, 83, 84, 85, 86, 87, 88, 89, 90, 91, 92, 93, 94, 95, 96, 97% sequence identity to PtUGT1. In one most preferred embodiment, the amino acid sequences of the polypeptide having glycosyltransferase enzyme activity is SEQ ID NO 4. In another most preferred embodiment, the amino acid sequences of the polypeptide having glycosyltransferase enzyme activity is SEQ ID NO 6. In another most preferred embodiment, the amino acid sequences of the polypeptide having glycosyltransferase enzyme activity is SEQ ID NO 8.

A person skilled in the art will be familiar with methods of providing the enzymes. Such enzymes may for example be microbially produced—such as recombinantly or by natural producers, or be synthesized.

In one embodiment the nucleotide sugar is a UPD-sugar, such as UDP-glucose, UPD-rhamnose, UPD-xylose, and UPD-arabinose. In a preferred embodiment, the nucleotide sugar is UDP-glucose. UDP-glucose may be provided directly as UDP-glucose or indirectly in the form of other sugars or sugar-containing molecules which are then converted into UDP-glucose. One example of such indirect providing of UPD-glucose is by providing sucrose along with UDP, which then by enzymatic catalysis (such as using Sucrose synthase (SuSy) EC 2.4.1.13) is converted to UDP-glucose.

Indoxyl may in the presence of oxygen react with the oxygen—such as in competition with the enzymatic glycosylation reaction. It is therefore an advantage to provide an oxygen reduced, oxygen free, or substantially oxygen free environment for the glycosylation reaction to take place, to ensure the indoxyl does not spontaneously dimerize.

In one embodiment, the reaction conditions for glycosylation of indoyl to form indican is substantially oxygen free. In one embodiment, the reaction conditions comprises less than 2% free oxygen, such as less than 1.5, 1, 0.5, or even less than 0.1% free oxygen and/or is maintained as a pressure less than 10, 9, 8, 7, 6, 5, 4, 3, 2 kPa or even less than 1 kPa, to reduce likelihood of oxidizing the reactive group of the compound—i.e. to reduce likelihood of the indoxyl spontaneously dimerizing to form indigo.

The indoxyl is preferably incubated with the glycosyltransferase enzyme at temperature and pH conditions optimal for the enzyme, as a person skilled in the art would recognize and routinely optimize.

II.ii. Aqueous Solution Comprising Indican

The indican provided in step (a) of the method for producing indigo from indican, and the method of dyeing a product with indigo, is provided in an aqueous solution. In a preferred embodiment, the aqueous solution is neutral or acidic. These conditions provides the most clean conversion of indican to indigo with little or no by-product formation. In one embodiment, the indican solution has a pH of or lower than 8.0, 7.5, 7.0, 6.5, 6.0, 5.5, 5.0, 4.5, 4.0, 3.5, 3.0, 2.5, 2.0, 1.5 or even lower than 1.0. In one embodiment, the indican solution has a pH in the range of 1-8, 1-7, 1-6, 1-5, 1-4, or 1-3. In one embodiment, the aqueous solution is an acidic solution of HCl, CH3COOH, CF3COOH, HNO3, HCOOH, and H₂SO₄.

The concentration of indican in the aqueous solution for dyeing a product is preferably at least 20, 30, 40, 50, 60, 70, 80, 90, 100, 120, 140, 160, 180, 200, 250, 300, 350, 400, 450, 500, 550, 600, or even 650 mM indican, such as between 20-600, 20-500, 20-400, 20-300, 20-250, 20-200, 20-180, 20-160, 20-140, 20-120, 20-100, 20-90, 20-80, 20-70, 20-60, 20-40 mM or between 30-60 or 35-50 mM indican.

The preferred amount of indican in solution depends on the use of the final indigo product. In preferred embodiments, the acidic aqueous solution comprising indican is used in the process of dyeing a fabric, wherein the amount of indican used in the dyeing process is between 0.05-100, 0.1-50, 0.1-45, 0.1-40, 0.1-35, 0.1-30, 0.1-25, 0.1-20, 0.1-15, 0.1-10, 0.1-9, 0.1-8, 0.1-7, 0.1-6, 0.1-5, 0.1-4, 0.1-3, 0.1-2, 0.1-1 μmol/cm² fabric, or between 0.2-5, 0.3-5, 0.4-5, 0.5-5, 0.6-5, 0.7-5, 0.8-5, 0.9-5, 1-5, 1.5-5, 2-5, 2.5-5, 3-5, 3.5-5, 4-5, 5.5-5 μmol/cm² fabric, or between 0.5-1.5, 0.3-2, 0.1-4, 0.05-10, or 0.1-10 μmol/cm² fabric.

II.iii. Light Exposure

Light sources and preferred wavelength are disclosed above. The light exposure preferably takes place in wet conditions—i.e. the conversion of indican to indigo takes place in the aqueous solution.

In some preferred embodiments, an indican solution is applied to a product by mixing, submerging, soaking, or coating the product in the indican solution, and the product is then exposed to light while in the indican solution. In other words, the indican-treated product is preferably not dried prior to light exposure. In one further embodiment, shaking, stirring or other means of mixing is preferably applied during the light exposure, to ensure the desired ring-pattern in the photochemical coloring of the fabric.

In one embodiment, where the light source is natural sunlight, the indican-treated product intended for dyeing is simply placed in natural sunlight, such as in the open outdoors.

In some embodiments, the indican-treated product is exposed to light by bringing a light-source into close proximity to the fluidic structure with or without the use of lenses or other optical systems, where the position, light intensity and duration of exposure is controlled by a person skilled in the art. The total light (radiant) exposure of a selected product will depend on the light source applied, the distance between the light source and the product, and the total time of exposure.

In one embodiment, the indican-treated product is irradiated by a light source, wherein said product received an average of between 20-220, 30-220, 40-220, 50-220, 60-220, 70-220, 80-220, 90-220, 100-220, 110-220, 120-220, 130-220, 140-220, 150-220, 160-220, 170-220, 180-220, 190-220, 200-220, 210-220 W/m2, or between 20-30, 20-40, 20-50, 0-60, 20-70, 20-80, 20-90, 20-100, 20-110, 20-120, 20-130, 20-140, 20-150, 20-160, 20-170, 20-180, 20-190, 20-200, 20-210 W/m2. In one embodiment, the indican-treated product is irradiated by a light source, wherein said product received an average of between 20-220, between 30-210, between 40-200, between 50-190, between 60-180, between 70-170, between 80-160, between 90-150, between 100-140, or between 110-130 W/m2. In some embodiments, the indican-treated product is irradiated by a light source, wherein said product receives between 37-208 W/m², between 65-208 W/m², or between 109-168 W/m².

In one embodiment, the indican-treated product is exposed to the light source for at least 5, 15, 30, 60, 90, 120, 150, 180, 210, 240, 270, 300 or even 360 minutes, such as exposed for a time period of between 5-60 minutes, 30-120 minutes, 90-240 minutes, or 120-360 minutes.

In one embodiment, the final radiant exposure of the indican-treated product is between 100-10000, 500-10000, 1000-10000, 1500-10000, 2000-10000, 2500-10000, 3000-10000, 3500-10000, 4000-10000, 4500-10000, 5000-10000, 5500-10000, 6000-10000, 6500-10000, 7000-10000, 7500-10000, 8000-10000, 8500-10000, 9000-10000, 9500-10000 J/m², or between 100-500, 100-1000, 100-1500, 100-2000, 100-2500, 100-2500, 100-3000, 100-3500, 100-4000, 100-4500, 100-5000, 100-5500, 100-6000, 100-6500, 100-7000, 100-7500, 100-8000, 100-8500, 100-9000, 100-9500.

In some embodiments, the final radiant exposure of the indican-treated product is 100-10000, 250-5000, 500-3000, 600-2000, 700-1500 kJ/m². In some embodiments, the final radiant exposure of the indican-treated product is 100-10000 kJ/m², 600-1000 kJ/m², or 1000-2000 J/m². Too little radiant exposure may lead to too weak coloring of the product, while too much radiant exposure may lead to off-coloring, such as caused by formation byproducts, or possibly even destroy the formed indigo.

### II.iv Detection of Intermediates and Final Product

Intermediates and compounds produced by the present invention may be detected by HLPC-UV, LC-MS, NMR, or similar analytical equipment as recognized by a person skilled in the art.

The CIELAB color space, also referred to as L*a*b*, is a color space defined by the International Commission on Illumination (abbreviated CIE). It expresses color as three values: L* for perceptual lightness, and a* and b* for the four unique colors of human vision: red, green, blue, and yellow. CIELAB is useful in industry for detecting differences in color. CIELAB values are measured using a benchtop spectrophotometer (MetaVue VS3200, X-Rite (USA)). The indigo color may be reported by its L*a*b* values.

### II.v Indigo Blue Color

In preferred embodiments, the indigo dye color obtained by the methods of the present invention resembles the standard blue indigo denim color. Preferably, the color of the final indigo dyed product of the invention has L* value 10-80, a* value −5-5, and b* value −20-0.

A person skilled in the art will recognize that the different feature of the light source mentioned above will affect the color development. The disclosed wavelength will excite the glycosidic bond in indican and break the bond whereby indigo is then spontaneously formed. The color intensity and purity will especially depend on total radiant exposure—which thereby will affect the final color of a dyed product.

### V. Use of Light in Denim Dyeing

In a further aspect, the present invention discloses the use of light for conversion of indican to indigo. In preferred embodiments, the wavelength of said light is between 300-450 nm, between 310-440 nm, between 320-430 nm, between 330-420 nm, between 340-410 nm, between 350-400 nm, between 360-390 nm, between 370-380 nm. In further preferred embodiments, the indigo product is simultaneously used for dyeing a product, such as a textile or fabric.

### VI. Advantages and Commercial Application

As discussed previously, and further evidenced in the below examples, the method of the present invention provides a greener and cheaper alternative to current fabric and textile dyeing processes. The fact that light, such as natural sun light, can be used to dye denim allows for a cheap (essentially free), sustainable, and low-tech denim dyeing. The main advantage is sustainability as there is no need for harsh chemicals or expensive enzymes. Also, indican can be applied on the fabric directly prior to light exposure, so the indigo is formed only on fabric, which alleviates the indigo in wastewater, another major environmental concern. Though most of the current dyeing processes take place in countries with low ecological regulations, may denim suppliers and uses have growing demand for more sustainable processes.

### Preferred Embodiments of the Invention

Preferred embodiment 1: A method for producing indigo, comprising the steps of
a. providing an aqueous solution comprising indican,
b. exposing the indican to a light source,
wherein the indican is exposed to sufficient radiant energy by the light source to convert at least 50% of the indican to indigo; and wherein the glycosidic bond in indican is cleaved solely by said light exposure.

Preferred embodiment 2: A method of dyeing a product with indigo, comprising the steps of
a. providing indican in an aqueous solution,
b. applying the indican solution from step (a) to said product, and
c. exposing the indican-treated product from step (b) to a light source to obtain a dyed product,
wherein the glycosidic bond in indican is cleaved solely by said light exposure.

Preferred embodiment 3: The method according to preferred embodiment 1 or 2, wherein the aqueous solution in step (a) is acidic.

Preferred embodiment 4: The method according to any one of preferred embodiments 1-3, wherein the light source is selected from sun light, artificial sunlight bulb, broadband light source, LED light.

Preferred embodiment 5: The method according to any one of preferred embodiments 1-4, wherein the wavelength of said light source is 300-470 nm.

Preferred embodiment 6: The method according to any one of preferred embodiments 2-5, wherein the indican-treated product is irradiated with 20-220 W/m² by said light source.

Preferred embodiment 7: The method according to any one of preferred embodiments 2-6, wherein the indican-treated product is exposed to the light source for up to 300 minutes.

Preferred embodiment 8: The method according to any one of preferred embodiments 2-7, wherein the final radiant exposure of the indican-treated product is 100-10000 J/m².

Preferred embodiment 9: The method according to any one of preferred embodiments 2-8, wherein the light exposure takes place while the product is submerged or soaked in the indican solution.

Preferred embodiment 10: The method according to any one of preferred embodiments 2-9, wherein the product is selected from one or more of a yarn, textile, and fabric, preferably cotton-based product.

Preferred embodiment 11: The method according to any one of preferred embodiments 2-10, wherein the product is textile or fabric, and wherein the concentration of indican in the indican solution is 0.1-10 μmol/cm² of textile or fabric.

Preferred embodiment 12: The method according to any one of preferred embodiments 1-11, wherein step (a) comprises: providing (i) indoxyl, (ii) a nucleotide sugar, and (iii) a polypeptide having glycosyltransferase enzyme activity, mixing components (i), (ii) and (iii), preferably at reaction conditions wherein less than 2% free oxygen is present, and letting the mixture react to obtain indican.

Preferred embodiment 13: Use of light for conversion of indican to indigo.

Preferred embodiment 14: The use of light according to preferred embodiment 13, wherein said indigo is simultaneously used for dyeing a product.

Preferred embodiment 15: The use of light according to preferred embodiment 13 or 14, wherein the wavelength of said light is 300-450 nm.

### EXAMPLES

### General Methodology

CIELAB color space (L*, a*, and b*) values were obtained using a benchtop spectrophotometer (MetaVue VS3200, X-Rite (USA)). CIEL data from 60 fabric swatches prepared from commercially purchased blue denim jeans are shown as violin and boxplots, with the values (bullet shape) from different colored experimental fabric swatches for the present invention overlaid.

### Example 1: Denim Dyeing Using Sunlight Vs Enzyme

Sunlight exposure: 1-5 μmol/cm2 indican was dissolved in 100 μL/cm2 and added on ready-to-dye denim discs (kindly provided by Nudie Jeans, Sweden) of 20 cm2 (5.04 cm diameter, 798+/−2 mg) in a petri dish. The discs were exposed to sunlight-like light by using an artificial sunlight bulb (OSRAM, U VITALX 300W E), for 4 hours at room temperature, without stirring. The discs were then washed with water and detergent, dried, and analyzed by CIE.

Enzymatic treatment (beta-glucosidase): 1-5 μmol/cm2 indican was dissolved in 100 μL/cm2 and added on ready-to-dye denim discs of 20 cm2 (5.04 cm diameter, 798+/−2 mg) in a petri dish. 2 mg/cm2 of ScGlu (Secale cereale beta-glucosidase) in 50 μL/cm2 of 50 mM phosphate-citrate buffer pH 9 (final concentration 16.7 mM) was added, and the enzymatic reaction proceeded for 4 h at room temperature, in the dark, without stirring. The discs were then washed with water and detergent, dried, and analyzed by CIE.

CEIL values for the dyed textiles are shown in Table 1.

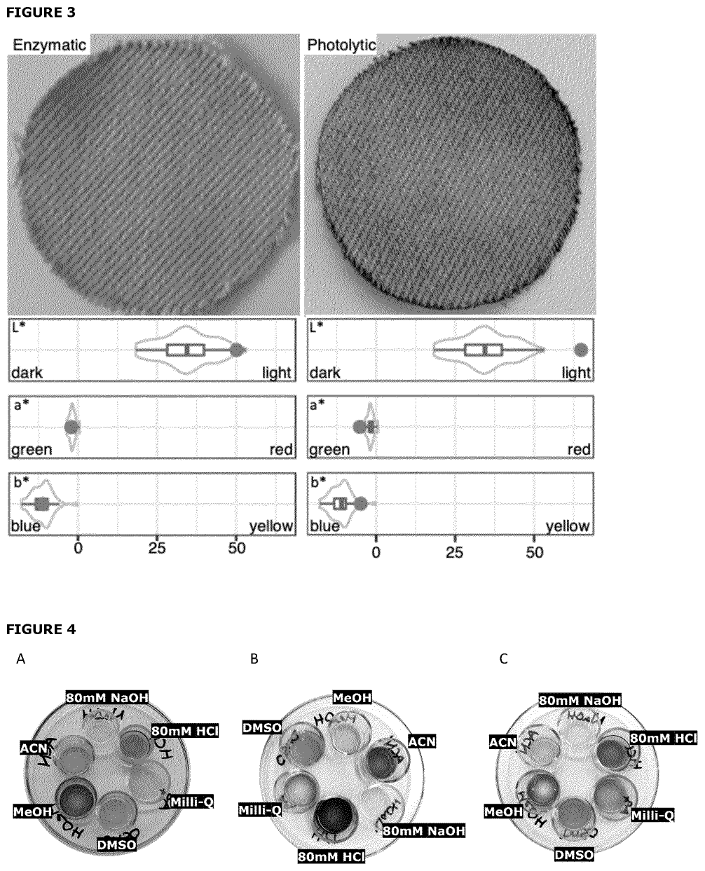

TABLE 1
CIEL values for dyed textiles

| sample | Treatment of indican | L* | a* | b* |
|---|---|---|---|---|
| B31 stripped | Sunlight-like exposure | 58.87 | −3.71 | −11.31 |
| B31 non stripped | Sunlight-like exposure | 59.06 | −4.31 | −9.83 |
| B35 stripped | Sunlight-like exposure | 51.88 | −3.23 | −8.77 |
| B35 non stripped | Sunlight-like exposure | 55.34 | −5.46 | −8.51 |
| B36 stripped | Sunlight-like exposure | 50.14 | −2.09 | −11.16 |
| B36 non stripped | Sunlight-like exposure | 46.88 | −2.1 | −11.49 |
| B58C | LED light exposure (365 | 64.85 | −5.21 | −4.91 |
| (FIG. 3 right) | nm, Thorlabs, Inc., | | | |
| | M660L4-C1) | | | |
| BGL1 stripped | Beta-glucosidase | 70.62 | −6.41 | −7.22 |
| BGL1 non stripped | Beta-glucosidase | 73.97 | −6.10 | −4.58 |
| BGL2 stripped | Beta-glucosidase | 54.01 | −6.05 | −13.8 |
| BGL2 non stripped | Beta-glucosidase | 60.05 | −7.65 | −7.93 |
| BGL3 stripped | Beta-glucosidase | 50.1 | −2.1 | −11.2 |
| (FIG. 3 left) | | | | |

Conditions for each B#.
B31: indican conc. = 1 μmol/cm2, volume = 0.23 ml/cm2, lid on container = yes, mixing source = magnet, exposure time = 6 hrs., repeats = 1, lamp distance = 30 cm.
B35: indican conc. = 6.8 μmol/cm2, volume = 0.23 ml/cm2, lid on container = yes, mixing source = magnet, exposure time = 6 hrs., repeats = 1, lamp distance = 30 cm.
B36: indican conc. = 1 μmol/cm2, volume = 0.23 ml/cm2, lid on container = yes, mixing source = magnet, exposure time = 6 hrs., repeats = 2, lamp distance = 30 cm.
B58C: indican conc. = 1 μmol/cm2, volume = 0.45 ml/cm2, lid on container = no, mixing source = shaking table, exposure time = 2 hrs., repeats = 1, lamp distance = 55 cm.

The feasibility of photocleavage of indican for formation of indigo is further evidenced in FIG. 3, showing a picture as well the respective CEIL value of a fabric swatch dyed by the enzymatic process (sample B36 front side) compared a fabric swatch dyed by the photolytic process of the present invention (sample BGL2 front side). Satisfactory blue denim color is indeed obtained by the simple photolytic process.

### Example 2: Testing Different Light Sources and Solvents

Indican (5 mg, 16.93 μmol) was dissolved in a selected solvent (520 μL) in a glass container which was then closed with a glass lid. The closed container was then placed under a selected light source for 2 hours, before analysis by LCMS. The tested solvents were: 80 mM NaOH (aq), 80 mM HCl (aq), milli-Q water, dimethyl sulfoxide (DMSO), methanol (MeOH) and acetonitrile (ACN). The tested light sources were: a mercury UV bulb (Omnilux, 89514005), a household bulb (DURA LAMP, 07018), and an artificial sunlight bulb (OSRAM, U VITALX 300W E).

The results are shown in FIG. 4 and table 2. Interesting, a range of different colors were observed depending on the solvent used. HCl was found to be a good solvent candidate for obtaining the desired blue denim color by photocleavage of indican to indigo.

TABLE 2
Color observations using different solvents and light sources

| Solvent | Light source | Color - visual observation |
|---|---|---|
| NaOH (aq) | mercury UV bulb | No color |
| NaOH (aq) | household bulb | Light pink |
| NaOH (aq) | artificial sunlight bulb | Light pink |
| HCl (aq) | mercury UV bulb | Light blue |
| HCl (aq) | household bulb | Dark blue |
| HCl (aq) | artificial sunlight bulb | Dark blue |
| milli-Q water | mercury UV bulb | Light pink |
| milli-Q water | household bulb | Blue |
| milli-Q water | artificial sunlight bulb | Purple |
| dimethyl sulfoxide | mercury UV bulb | Yellow |
| dimethyl sulfoxide | household bulb | Yellow |
| dimethyl sulfoxide | artificial sunlight bulb | Yellow |
| methanol | mercury UV bulb | Dark green |
| methanol | household bulb | Light green |
| methanol | artificial sunlight bulb | Dark turquoise |
| acetonitrile | mercury UV bulb | Light green |
| acetonitrile | household bulb | Dark green |
| acetonitrile | artificial sunlight bulb | Light green |

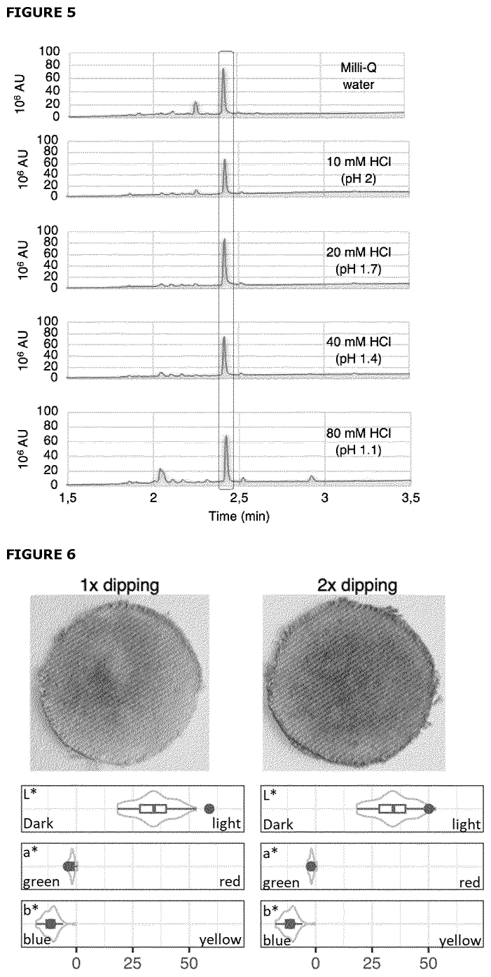

In further optimizing the purity of the product, different HCl concentrations were tested. The product profiles were measured and analyzed by LCMS using Waters AQUITY UPLC system equipped with PDA and a SQD electrospray MS detector. Column: Thermo accucore C18 2.6 μm, 2.1×50 mm. Column temp: 50° C. Flow rate: 0.6 mL/min. Solvent A1—0.1% formic acid in water, Solvent B1—0.1% formic acid in ACN. Gradient: 5% B to 100% B in 3 min., hold 0.1 min., total run time 5 min. The chromotograms are shown in FIG. 5. LCMS was used to quantify the consumption of indican.

Varying pH between 1 and 2 (using HCl as solvent) identified pH 1.7 (20 mM HCl (aq)) as optimal, such as in regard to minimal byproduct formation.

The annotated (boxed) peak was identified as indigo by NMR, and color extracted from commercial denim also gave rise to a peak with identical retention time (data not shown).

### Example 3: Fabric Dyeing

### 3.1 Repeated Dipping

Artificial sunlight bulb (mimicking natural sunlight) was used for photocleavage of indican and dyeing of fabric. Reaction conditions: 1 μmol indican/(cm2 fabric), 0.225 mL 20 mM HCl (aq)/(cm2 fabric), magnet for stirring, 6 hours exposure time. Results are provide in FIG. 6 (pictures of dyed fabric) and Table 3 (CIEL values of dyed fabric). The picture and the CIELAB values demonstrate that repeated dipping allows for dynamic coloring analogous to the conventional dyeing process.

TABLE 3
CIEL values for dyed textiles

| | 1x dipping | 2x dipping |
|---|---|---|
| L* | 58.9 | 50.1 |
| b* | −11.3 | −11.2 |
| a* | −3.7 | −2.1 |

### 3.2 Different Mixing Methods

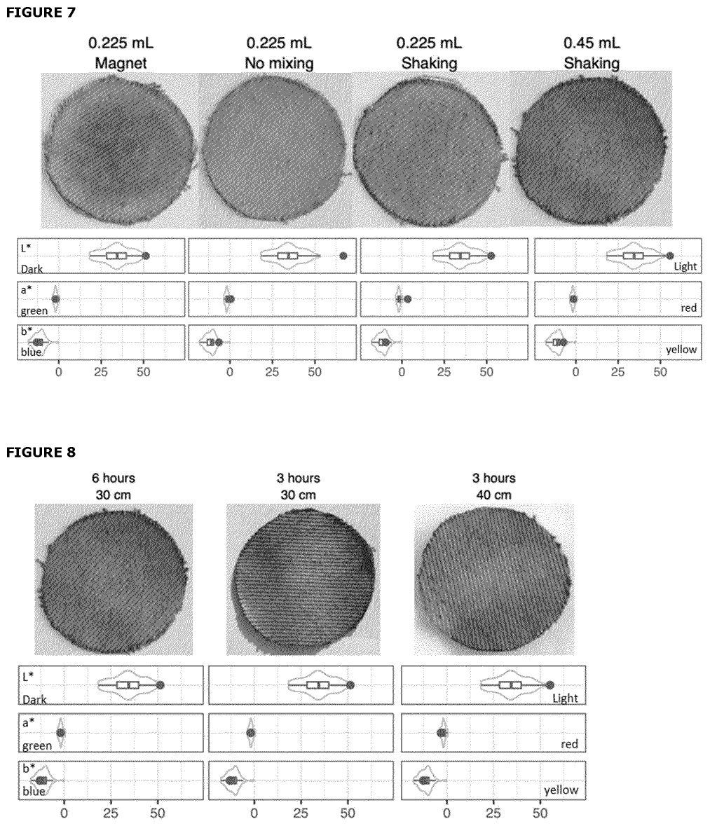

Artificial sunlight bulb was used for photocleavage of indican and dyeing of fabric. Reaction conditions: 1 μmol indican/(cm² fabric), 2× dipping in 20 mM HCl (aq), 6 hours exposure time. Results are provide in FIG. 7 (pictures of dyed fabric) and Table 4 (CIEL values of dyed fabric).

TABLE 4
CIEL values for dyed textiles

| | 0.225 mL | 0.225 mL | 0.225 mL | 0.45 mL |
|---|---|---|---|---|
| | solvent | solvent | solvent | solvent |
| | Magnet | No mixing | Shaking | Shaking |
| L* | 50.1 | 66.7 | 52.6 | 55.7 |
| b* | −11.2 | −6.5 | −9.4 | −7.4 |
| a* | −2.1 | 0.5 | 3.4 | −1.4 |

The data demonstrates that mixing mode has an influence on color development, with a magnet giving rise to uneven coloring, where the distribution depends on the magnet position. No mixing leads to a lighter color, i.e. less efficient use of indican. Shaking gives rise to even and efficient color development but promotes byproduct formation (red, likely due to excess aeration), which can be alleviated by increasing the liquid volume, which is given here per square centimeter of fabric.

### 3.3 Exposure Time and Illumination Area

Artificial sunlight bulb was used for photocleavage of indican and dyeing of fabric. Reaction conditions: 1 μmol indican/(cm2 fabric), 0.45 mL 20 mM HCl (aq)/(cm2 fabric), Shaking for mixing method, 2× dipping. Results are provide in FIG. 8 (pictures of dyed fabric) and Table 5 (CIEL values of dyed fabric).

TABLE 5
CIEL values for dyed textiles

| | 6 hours, | 3 hours, | 3 hours, |
|---|---|---|---|
| | 30 cm distance | 30 cm distance | 40 cm distance |
| L* | 55.7 | 51.3 | 55.2 |
| b* | −7.4 | −13.0 | −12.6 |
| a* | −1.4 | −2.0 | −3.2 |

The data demonstrates that decreasing the exposure time to half and increasing lamp distance to allow a larger area to be illuminated did not compromise color quality.

### Example 4: Wavelength Optimization

Testing 10 LEDs at different wavelengths allowed for the identification of the optimal wavelength for photolytic cleavage of indican.

To identify which specific wavelengths are responsible for initiating the photolytic process, a series of 10 LEDs with specific wavelengths of 308, 325, 340, 365, 415, 470, 530, 590, 625, and 660 nm were tested. The LEDs was purchased from Thorlabs, Inc., 308 nm (M310L1), 325 nm (M325L5), 340 nm (M340L4), 365 nm (M365L2), 415 nm (M415L4), 470 nm (M470L5), 530 nm (M530L4), 590 nm (M590L4), 625 nm (M625L4), 660 nm (M660L4).

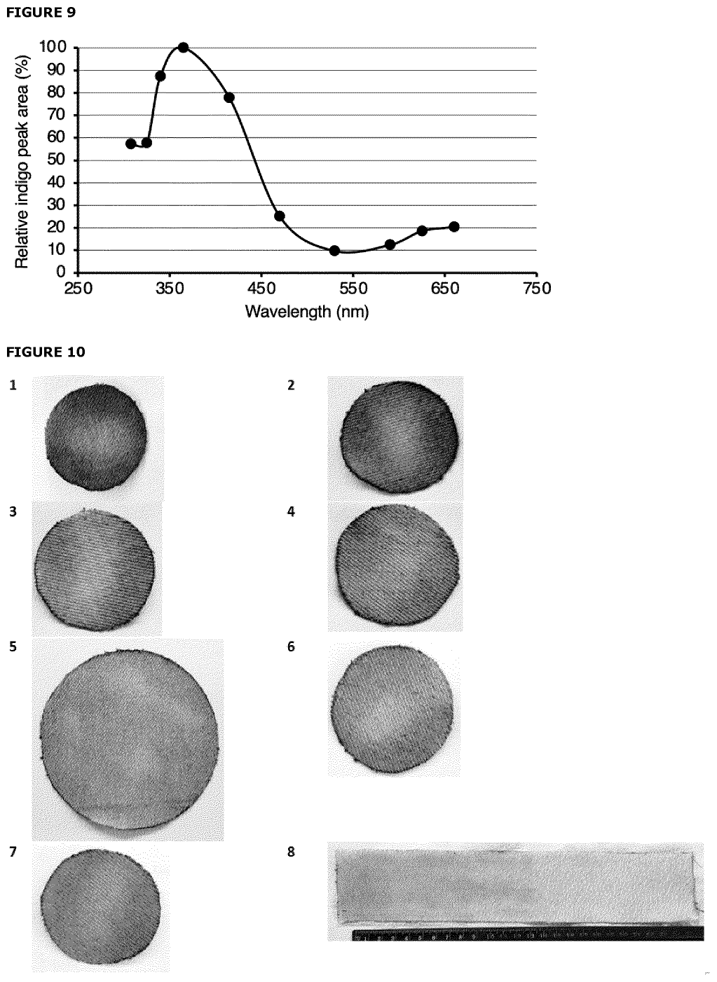

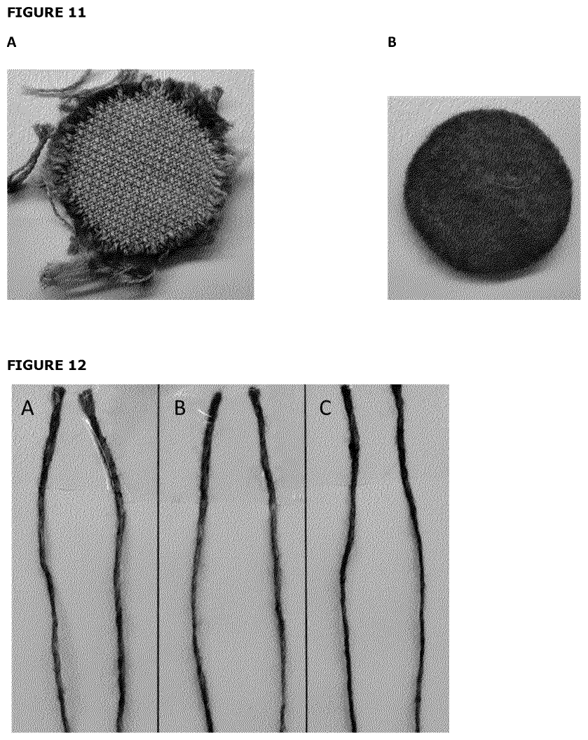

Specifically, 5 mg indican was dissolved in 520 μL 20 mM HCl (aq) in a glass container covered with a quartz-glass lid and placed 14 cm under the LED. Samples were exposed to light for 2 hours before analysis by LCMS. It was found that 365 nm wavelength gave the highest conversion of indican to indigo; the results are presented in FIG. 9.

### Example 5: Use of Collimator

Using the optimal wavelength identified above, further optimization of the photolytic reaction was performed. The light source was a single diode (365 nm) modified with an Olympus BX and IX, collimator (50 mm²) from Thorlabs, Inc. (Olympus BX & IX+365 nm LED(M660L4-C1)).

Reaction conditions and results are found in table 10 and FIG. 10.

TABLE 10
Reaction conditions and results

| | Reaction conditions | | | | | | | | |
|---|---|---|---|---|---|---|---|---|---|
| | Distance | | | | | | | | |
| | between | | | | | | | | |
| | fabric | | | | | | | | |
| Test | and lens | Irradiance¹ | Exposure | Fabric | Other | | CEIL values | | |
| # | (cm) | (W/m²) | time | area | info | Visual observation | L* | b* | a* |
| 1 | 14 | ND | 2 × | 20 cm² | | The brownish spot in the middle is due the lens | 59.91 | −1.72 | 3.46 |
| | | | 3 hrs | | | being too close to the fabric (becoming “burned”) | | | |
| 2 | 30 | Approx. | 2 × | 20 cm² | | The brownish spot is still being formed | 59.93 | −2.24 | 4.15 |
| | | 980 | 3 hrs | | | and too high amounts of indigo was produced | | | |
| | | | | | | (LED lens is much more efficient | | | |
| | | | | | | than the artificial sunlight bulb) | | | |
| 3 | 30 | Approx. | 1 × | 20 cm² | | The brownish spot is still being formed | 70.05 | −1.31 | 4.6 |
| | | 980 | 3 hrs | | | | | | |
| 4 | 55 | Approx. | 1 × | 20 cm² | | This gave a beautiful colour dept and without | 65.17 | −4.13 | −7.95 |
| | | 190 | 3 hrs | | | the brownish spot | | | |
| 5 | 55 | Approx. | 1 × | 85 cm² | | A satisfying colour and dept was achieved | 62.94 | −5.02 | −9.69 |
| | | 190 | 3 hrs | | | | | | |
| 6 | 55 | Approx. | 1 × | 20 cm² | no | A satisfying colour and dept was achieved | 64.85 | −5.21 | −4.91 |
| | | 190 | 3 hrs | | lid | | | | |
| 7 | 55 | Approx. | 1 × | 20 cm² | no | Satisfied with the colour and dept, | 76.95 | −5.76 | −5.94 |
| | | 190 | 2 hrs | | lid | we investigated the maximum area we | | | |
| | | | | | | could colour with one LED lens | | | |
| 8 | 55 | Approx. | 1 × | Rectangular | no | With this it is concluded that one LED lens | 78.7 | −6.84 | −6.54 |
| | | 190 | 2 hrs | piece | lid | can colour a radius of 13 cm (area of 530.9 cm2) | | | |
| | | | | 26.7 × | | CIE values within this area are constant | | | |
| | | | | 5.2 cm | | | | | |

¹Irradiance was measured using a spherespectroradiometer (Instrument systems, ISP150 and CAS140D).

### Example 6: Other Fabric Materials

Following the procedures described in example 3 and 4, a sample of 90/10 wool/nylon (STEELOUT) and 100% wool (DIVINA) respectively, were dyed.

Specifically, 2.46 μmol indican/(cm² fabric) was dissolved in 1.13 mL 20 mM HCl (aq)/(cm² fabric) in a glass container a little wider than the fabric diameter and placed in a 7 cm distance under the LED. Samples were exposed to light for 3 hours; the results are presented in FIG. 10 (A: STEELOUT front and B: DIVINA front).

### Example 7: Yarn Coloring

The procedure from example 3 and 4 was redesigned to color yarn samples. Solutions of indican were prepared in dark glass containers and always kept in a dark box. The yarn was cut in a 15 cm samples size and soaked in the solutions above before being moved to a glass container with 20 mM HCl and exposed to the LED (365 nm). Different conditions were tested; (1) the solvent of the soaking solution (20 mM HCl or milliQ water), (2) the concentration of indican in the soaking solution (7 mg/mL, 5 mg/mL, 3 mg/mL, 2 mg/mL, 1 mg/mL), (3) the time soaking (1 hr., 2 hrs., 3 hrs., 6 hrs.), (4) the time of light exposure (1 hr., 2 hrs., 3 hrs.) and (5) the number of repeats (1 time, 2 times, 3 times).

One specific example: the soaking solution was prepared by dissolving indican (5 mg/mL) in milliQ water (5 mL) under dark conditions. Two samples of yarn (15 cm) were soaked in the solution for 3 hrs., before being moved to glass containers (4 cm in diameter) with 20 mM HCl (4 mL) and exposed to the LED (365 nm) for 3 hrs. For multiple repeats the yarn was rinsed before returning to the soaking solution container, and the rest as described above. Three examples can be seen in FIG. 12.

### Example 6: Providing Indican by Glycosylation of Indoxyl

Synthesis of indican from Indoxyl acetate by glycosyl transferase (GT) was demonstrated using glycosyl transferase having SEQ ID NO 4 (PtUGT1 mut87).

Synthesis of indican from high concentrations of indoxyl-acetate (100 mM) was performed in triplicate inside an anaerobic chamber, using glass HPLC vials stirred with small magnets and at 30° C. Reaction consisted on 3.5 mg indoxyl-acetate, 90 mM buffer phosphate-citrate pH8, 1 mM UDP, 200 mM sucrose, 2U of Esterase from Bacillus subtilis (Sigma Aldrich), and different concentrations of GT/SuSy always at a molar ratio of 1:5 (50 μg, 20 μg, 10 μg, 5 μg for GT; and 432.5 μg, 173 μg, 86.7 μg, 43.3 μg for SuSy). Sucrose synthase (SuSy) converts sucrose and uridine 5′-diphosphate (UDP) into UDP-glucose. The reaction was started by the addition of all three enzymes (Esterase, GT and SuSy) and the progression was followed by HPLC. Samples were collected at 1, 2, 3, 6, 12, 24, and 32 hours.

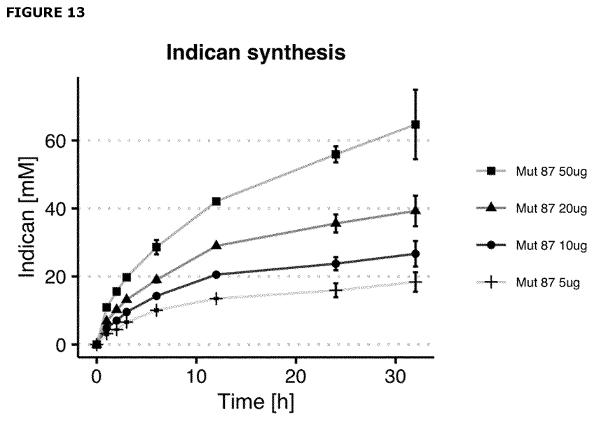

FIG. 13 shows that GT enzyme efficiently produces indican from indoxyl acetate.

1. A method for producing indigo, comprising the steps of
a. providing an aqueous acidic solution comprising indican, and
b. exposing the indican to a light source, wherein the wavelength of said light source is 300-470 nm,
and wherein conversion of indican to indigo is mediated solely by said light exposure.

2. The method according to claim 1, wherein the indican is exposed to sufficient radiant energy by the light source to convert at least 50% of the indican to indigo.

3. A method of dyeing a product with indigo, comprising the steps of
a. providing indican in an acidic aqueous solution,
b. applying the indican solution from step (a) to said product, and
c. exposing the indican-treated product from step (b) to a light source to obtain a dyed product,
wherein the wavelength of said light source is 300-470 nm, and
wherein the glycosidic bond in indican is cleaved solely by said light exposure.

4. The method according to any one of claims 1-3, wherein the light source is selected from sun light, artificial sunlight bulb, broadband light source, and LED light.

5. The method according to claim 3 or 4, wherein the indican-treated product is irradiated with 20-220 W/m² by said light source.

6. The method according to any one of claims 3-5, wherein the indican-treated product is exposed to the light source for up to 300 minutes.

7. The method according to any one of claims 3-6, wherein the final radiant exposure of the indican-treated product is 100-10000 J/m².

8. The method according to any one of claims 3-7, wherein the light exposure takes place while the product is submerged or soaked in the indican solution.

9. The method according to any one of claims 3-8, wherein the product is selected from one or more of a yarn, textile, and fabric, preferably cotton-based product.

10. The method according to any one of claims 3-9, wherein the product is textile or fabric, and wherein the concentration of indican in the indican solution is 0.1-10 μmol/cm² of textile or fabric.

11. The method according to claim 9 or 10, wherein the product is submerged, soaked, or coated with the indican solution.

12. The method according to any one of claims 1-11, wherein step (a) comprises: providing (i) indoxyl, (ii) a nucleotide sugar, and (iii) a polypeptide having glycosyltransferase enzyme activity, mixing components (i), (ii) and (iii), preferably at reaction conditions wherein less than 2% free oxygen is present, and letting the mixture react to obtain indican.

13. Use of light for conversion of indican to indigo, wherein the wavelength of said light is 300-450 nm, and wherein said indican is in an aqueous acidic solution.

14. The use of light according to claim 13, wherein said indigo is simultaneously used for dyeing a product.

15. The use of light according to claim 14, wherein the product is selected from one or more of a yarn, textile, and fabric.


SEQUENCE LISTING (8 sequences)

SEQ ID NO: 1 - DNA, 1437 bp, Persicaria tinctoria
  source 1..1437: mol_type = genomic DNA; note = Sequence encoding UDP-dependent glycosyltransferase (PtUGT1)
  CDS 1..1437: protein_id = 2; translation = MSESPAAPPTTAPPPHVIIVPSAGMGHLIPLAEFAKRLLPRFTF TFAVPTSGPPSSSQRDFLSSLPASIDTSFLPEVDLSDAPSDAQIETLMSLMVVRSLPS LRDLIASYSASGRRVAALVVDLFATDAIDVALELGIRPFIFFPSTAMTLSFFLHLEKL DETVSCEFAELSDPVQIPGCIPVHGKDLIDPVQDRKNDAYKWLLHHSKRYKLAEGVIV NSFEGLEGGPIRELLHPEPGKPRVYPVGPLIQAGSCEKGAAARPECLKWLDQQPRGSV LFVNFGSGGVLSTEQQNELAGVLAHSQQRFLWVVRPPNDGIANATYFSVDGEIDPLKL LPEGFLEQTAGRGLVLPMWAPQIDVLSHESTGGFLTHCGWNSTLESVFHGVPLITWPL YAEQKMNAVMLTEGLRVGLRPSVGKDGIIRGAEIARVIGELMEGEEGKRIRSKMQELK RAASAVLSKDGSSTRALEEVAKIWESKV
atgtctgaatcccccgccgctccaccaaccaccgctccaccaccgcacgtcataatcgtgccctccgccggcatgggccacctcatccccctcgccgagttcgccaagcgcctccttccgcgcttcaccttcaccttcgccgtacccaccagcggcccgccctcatcctcccagcgcgacttcctctcctccctccctgcctccatcgacacctccttcctccccgaggtcgacctctccgacgccccctccgacgcccaaatcgagactctcatgtccctcatggttgtccgctccctcccctcgctccgcgacctcattgcctcctactccgcctccggccgccgcgtcgccgccctcgtcgtcgaccttttcgccactgatgcaatcgacgtcgcccttgagctcggcatccgccctttcatcttcttcccctccaccgccatgaccctctccttcttcctccacctcgagaagcttgatgaaacggtgtcatgtgagtttgccgagctgtccgacccggttcagatccccgggtgtattccggtccacggcaaggatttgatcgacccggttcaggataggaagaacgacgcctacaagtggctcctccaccactccaagaggtataaattggccgagggtgttatcgtaaatagcttcgagggtttggaggggggaccgatcagggagcttttgcaccccgagccgggaaagccgcgggtttacccggtcggaccgctgattcaggccggttcgtgcgagaagggggcagctgcccggcctgagtgcttgaagtggttggaccagcagccacgtggatccgtcctattcgtgaatttcgggagtggtggggtcctcagtacggagcagcagaacgagcttgcaggtgtgctggcccacagccagcagaggttcctatgggtggttaggcctccaaacgacggcattgccaacgccacgtatttcagcgtcgacggggagatcgacccgttgaaactcctgcccgaggggttcttggagcagaccgcgggcaggggtttggtcttgccaatgtgggccccgcagatcgatgtcttaagccatgagtcgacgggcgggttcttgacgcattgtgggtggaattcaacactggagagcgtgttccatggggtaccactaattacatggcccctctatgcagagcaaaagatgaacgctgttatgctaaccgagggcctgagggtgggactcagaccctcagtgggtaaggatggaatcatccgaggtgctgagatcgcacgagttataggggagttgatggaaggtgaggaagggaaacgaatacggagtaagatgcaggagttgaagcgtgcggcttctgctgtattgagcaaagatggatcatctactcgagctcttgaagaggttgcaaaaatttgggaaagcaaggtttaa

SEQ ID NO: 2 - protein, 478 aa, Persicaria tinctoria
MSESPAAPPTTAPPPHVIIVPSAGMGHLIPLAEFAKRLLPRFTFTFAVPTSGPPSSSQRDFLSSLPASIDTSFLPEVDLSDAPSDAQIETLMSLMVVRSLPSLRDLIASYSASGRRVAALVVDLFATDAIDVALELGIRPFIFFPSTAMTLSFFLHLEKLDETVSCEFAELSDPVQIPGCIPVHGKDLIDPVQDRKNDAYKWLLHHSKRYKLAEGVIVNSFEGLEGGPIRELLHPEPGKPRVYPVGPLIQAGSCEKGAAARPECLKWLDQQPRGSVLFVNFGSGGVLSTEQQNELAGVLAHSQQRFLWVVRPPNDGIANATYFSVDGEIDPLKLLPEGFLEQTAGRGLVLPMWAPQIDVLSHESTGGFLTHCGWNSTLESVFHGVPLITWPLYAEQKMNAVMLTEGLRVGLRPSVGKDGIIRGAEIARVIGELMEGEEGKRIRSKMQELKRAASAVLSKDGSSTRALEEVAKIWESKV

SEQ ID NO: 3 - DNA, 1434 bp, synthetic construct
  source 1..1434: mol_type = other DNA; note = Sequence encodes Mut87, a mutant enzyme derived from PtUGT1
  CDS 1..1434: protein_id = 4; translation = MSESPAAPPTTAPPPHVIIVPSAGMGHLIPLAEFAKRLLPRFTF TFAVPTSGPPSSSQRDFLSSLPASIDTSFLPPVDLSDAPSDAKIETLMSLMVVRSLPS LRDLIASYVASGRRVAALVVDLFATDAIDVALELGIRPFIFFPSTAMTLSFFLHLEKL DETVSCEFAELSDPVQIPGCIPVHGKDLLDPVQDRKNDAYKWLLHHSKRYKLAEGVIV NSFEDLEGGPIRELLHPEPGKPRVYPVGPLIQAGSCEKGAAARPECLKWLDQQPRGSV LFVNFGSGGVLSTEQQNELALGLAHSQQRFLWVVRPPNDGIANATYFSVDGEIDPLKL LPEGFLEQTAGRGLVLPMWAPQIDVLSHESTGGFLTHCGWNSTLESVFHGVPLIAWPL YAEQKMNAVMLTEGLRVGLRPKVGKDGIIRGAEIARVIKELMEGEEGKRIRSKMQELK RAASAVLSKDGSSTRALEEVAKIWESKV
atgtctgaatcccccgccgctccaccaaccaccgctccaccaccgcacgtcataatcgtgccctccgccggcatgggccacctcatccccctcgccgagttcgccaagcgcctccttccgcgcttcaccttcaccttcgccgtacccaccagcggcccgccctcatcctcccagcgcgacttcctctcctccctccctgcctccatcgacacctccttcctccccccggtcgacctctccgacgccccctccgacgccaaaatcgagactctcatgtccctcatggttgtccgctccctcccctcgctccgcgacctcattgcctcctacgtggcctccggccgccgcgtcgccgccctcgtcgtcgaccttttcgccactgatgcaatcgacgtcgcccttgagctcggcatccgccctttcatcttcttcccctccaccgccatgaccctctccttcttcctccacctcgagaagcttgatgaaacggtgtcatgtgagtttgccgagctgtccgacccggttcagatccccgggtgtattccggtccacggcaaggatttgctggacccggttcaggataggaagaacgacgcctacaagtggctcctccaccactccaagaggtataaattggccgagggtgttatcgtaaatagcttcgaggatttggaggggggaccgatcagggagcttttgcaccccgagccgggaaagccgcgggtttacccggtcggaccgctgattcaggccggttcgtgcgagaagggggcagctgcccggcctgagtgcttgaagtggttggaccagcagccacgtggatccgtcctattcgtgaatttcgggagtggtggggtcctcagtacggagcagcagaacgagcttgcactgggtctggcccacagccagcagaggttcctatgggtggttaggcctccaaacgacggcattgccaacgccacgtatttcagcgtcgacggggagatcgacccgttgaaactcctgcccgaggggttcttggagcagaccgcgggcaggggtttggtcttgccaatgtgggccccgcagatcgatgtcttaagccatgagtcgacgggcgggttcttgacgcattgtgggtggaattcaacactggagagcgtgttccatggggtaccactaattgcatggcccctctatgcagagcaaaagatgaacgctgttatgctaaccgagggcctgagggtgggactcagacccaaagtgggtaaggatggaatcatccgaggtgctgagatcgcacgagttataaaagagttgatggaaggtgaggaagggaaacgaatacggagtaagatgcaggagttgaagcgtgcggcttctgctgtattgagcaaagatggatcatctactcgagctcttgaagaggttgcaaaaatttgggaaagcaaggtt

SEQ ID NO: 4 - protein, 478 aa, synthetic construct
MSESPAAPPTTAPPPHVIIVPSAGMGHLIPLAEFAKRLLPRFTFTFAVPTSGPPSSSQRDFLSSLPASIDTSFLPPVDLSDAPSDAKIETLMSLMVVRSLPSLRDLIASYVASGRRVAALVVDLFATDAIDVALELGIRPFIFFPSTAMTLSFFLHLEKLDETVSCEFAELSDPVQIPGCIPVHGKDLLDPVQDRKNDAYKWLLHHSKRYKLAEGVIVNSFEDLEGGPIRELLHPEPGKPRVYPVGPLIQAGSCEKGAAARPECLKWLDQQPRGSVLFVNFGSGGVLSTEQQNELALGLAHSQQRFLWVVRPPNDGIANATYFSVDGEIDPLKLLPEGFLEQTAGRGLVLPMWAPQIDVLSHESTGGFLTHCGWNSTLESVFHGVPLIAWPLYAEQKMNAVMLTEGLRVGLRPKVGKDGIIRGAEIARVIKELMEGEEGKRIRSKMQELKRAASAVLSKDGSSTRALEEVAKIWESKV

SEQ ID NO: 5 - DNA, 1434 bp, synthetic construct
  source 1..1434: mol_type = other DNA; note = Sequence encodes Mut88, a mutant enzyme derived from PtUGT1
  CDS 1..1434: protein_id = 6; translation = MSESPAAPPTTAPPPHVIIVPSAGMGHLIPLAEFAKRLLPRFTF TFAVPTSGPPSSSQRDFLSSLPASIDTSFLPPVDLSDAPSDAKIETLMSLMVVRSLPS LRDLIASYVASGRRVAALVVDLFATDAIDVALELGIRPFIFFPSTAMTLSFFLHLEKL DETVSCEFAELSDPVQIPGCIPVHGKDLLDPVQDRKNDAYKWLLHHSKRYKLAEGVIV NSFEDLEGGPIRELLHPEPGKPRVYPVGPLIQAGSCEKGAAARPECLKWLDQQPRGSV LFVNFGSGGVLSTEQQNELALGLAHSQQRFLWVVRPPNDGIANATYFSVDGEIDPLKL LPEGFLEQTAGRGLVLPMWAPQIDVLSHESTGGFLTHCGWNSTLESVVHGVPLIAWPL YAEQKMNAVMLTEGLRVGLRPKVGKDGIIRGAEIARVIKELMEGEEGKRIRSKMQELK RAASAVLSKDGSSTRALEEVAKIWESKV
atgtctgaatcccccgccgctccaccaaccaccgctccaccaccgcacgtcataatcgtgccctccgccggcatgggccacctcatccccctcgccgagttcgccaagcgcctccttccgcgcttcaccttcaccttcgccgtacccaccagcggcccgccctcatcctcccagcgcgacttcctctcctccctccctgcctccatcgacacctccttcctccccccggtcgacctctccgacgccccctccgacgccaaaatcgagactctcatgtccctcatggttgtccgctccctcccctcgctccgcgacctcattgcctcctacgtggcctccggccgccgcgtcgccgccctcgtcgtcgaccttttcgccactgatgcaatcgacgtcgcccttgagctcggcatccgccctttcatcttcttcccctccaccgccatgaccctctccttcttcctccacctcgagaagcttgatgaaacggtgtcatgtgagtttgccgagctgtccgacccggttcagatccccgggtgtattccggtccacggcaaggatttgctggacccggttcaggataggaagaacgacgcctacaagtggctcctccaccactccaagaggtataaattggccgagggtgttatcgtaaatagcttcgaggatttggaggggggaccgatcagggagcttttgcaccccgagccgggaaagccgcgggtttacccggtcggaccgctgattcaggccggttcgtgcgagaagggggcagctgcccggcctgagtgcttgaagtggttggaccagcagccacgtggatccgtcctattcgtgaatttcgggagtggtggggtcctcagtacggagcagcagaacgagcttgcactgggtctggcccacagccagcagaggttcctatgggtggttaggcctccaaacgacggcattgccaacgccacgtatttcagcgtcgacggggagatcgacccgttgaaactcctgcccgaggggttcttggagcagaccgcgggcaggggtttggtcttgccaatgtgggccccgcagatcgatgtcttaagccatgagtcgacgggcgggttcttgacgcattgtgggtggaattcaacactggagagcgtggtccatggggtaccactaattgcatggcccctctatgcagagcaaaagatgaacgctgttatgctaaccgagggcctgagggtgggactcagacccaaagtgggtaaggatggaatcatccgaggtgctgagatcgcacgagttataaaagagttgatggaaggtgaggaagggaaacgaatacggagtaagatgcaggagttgaagcgtgcggcttctgctgtattgagcaaagatggatcatctactcgagctcttgaagaggttgcaaaaatttgggaaagcaaggtt

SEQ ID NO: 6 - protein, 478 aa, synthetic construct
MSESPAAPPTTAPPPHVIIVPSAGMGHLIPLAEFAKRLLPRFTFTFAVPTSGPPSSSQRDFLSSLPASIDTSFLPPVDLSDAPSDAKIETLMSLMVVRSLPSLRDLIASYVASGRRVAALVVDLFATDAIDVALELGIRPFIFFPSTAMTLSFFLHLEKLDETVSCEFAELSDPVQIPGCIPVHGKDLLDPVQDRKNDAYKWLLHHSKRYKLAEGVIVNSFEDLEGGPIRELLHPEPGKPRVYPVGPLIQAGSCEKGAAARPECLKWLDQQPRGSVLFVNFGSGGVLSTEQQNELALGLAHSQQRFLWVVRPPNDGIANATYFSVDGEIDPLKLLPEGFLEQTAGRGLVLPMWAPQIDVLSHESTGGFLTHCGWNSTLESVVHGVPLIAWPLYAEQKMNAVMLTEGLRVGLRPKVGKDGIIRGAEIARVIKELMEGEEGKRIRSKMQELKRAASAVLSKDGSSTRALEEVAKIWESKV

SEQ ID NO: 7 - DNA, 1434 bp, synthetic construct
  source 1..1434: mol_type = other DNA; note = Sequence encodes Mut90, a mutant enzyme derived from PtUGT1
  CDS 1..1434: protein_id = 8; translation = MSESPAAPPTTAPPPHVIIVPSAGMGHLIPLAEFAKRLLPRFTF TFAVPTSGPPSSSQRDFLSSLPASIDTSFLPPVDLSDAPSDAKIETLMSLMVVRSLPS LRDLIASYVASGRRVAALVVDLFATDAIDVALELGIRPFIFFPSTAMTLSFFLHLEKL DETVSCEFAELSDPVQIPGCIPVHGKDLLDPVQDRKNDAYKWLLHHSKRYKLAEGVIV NSFEDLEGGPIRELLHPEPGKPRVYPVGPLIQAGSCEKGAAARPECLKWLDQQPRGSV LFVNFGSGGVLSTEQQNELALGLAHSQQRFLWVVRPPNDGIANATYFSVDGEIDPLKL LPEGFLEQTAGRGLVLPMWAPQIDVLSHESTGGFLTHCGWNSTLESVVHGVPLICWPL YAEQKMNCVMLTEGLRVGLRPKVGKDGIIRGAEIARVIKELMEGEEGKRIRSKMQELK RAASAVLSKDGSSTRALEEVAKIWESKV
atgtctgaatcccccgccgctccaccaaccaccgctccaccaccgcacgtcataatcgtgccctccgccggcatgggccacctcatccccctcgccgagttcgccaagcgcctccttccgcgcttcaccttcaccttcgccgtacccaccagcggcccgccctcatcctcccagcgcgacttcctctcctccctccctgcctccatcgacacctccttcctccccccggtcgacctctccgacgccccctccgacgccaaaatcgagactctcatgtccctcatggttgtccgctccctcccctcgctccgcgacctcattgcctcctacgtggcctccggccgccgcgtcgccgccctcgtcgtcgaccttttcgccactgatgcaatcgacgtcgcccttgagctcggcatccgccctttcatcttcttcccctccaccgccatgaccctctccttcttcctccacctcgagaagcttgatgaaacggtgtcatgtgagtttgccgagctgtccgacccggttcagatccccgggtgtattccggtccacggcaaggatttgctggacccggttcaggataggaagaacgacgcctacaagtggctcctccaccactccaagaggtataaattggccgagggtgttatcgtaaatagcttcgaggatttggaggggggaccgatcagggagcttttgcaccccgagccgggaaagccgcgggtttacccggtcggaccgctgattcaggccggttcgtgcgagaagggggcagctgcccggcctgagtgcttgaagtggttggaccagcagccacgtggatccgtcctattcgtgaatttcgggagtggtggggtcctcagtacggagcagcagaacgagcttgcactgggtctggcccacagccagcagaggttcctatgggtggttaggcctccaaacgacggcattgccaacgccacgtatttcagcgtcgacggggagatcgacccgttgaaactcctgcccgaggggttcttggagcagaccgcgggcaggggtttggtcttgccaatgtgggccccgcagatcgatgtcttaagccatgagtcgacgggcgggttcttgacgcattgtgggtggaattcaacactggagagcgtggtccatggggtaccactaatttgctggcccctctatgcagagcaaaagatgaactgcgttatgctaaccgagggcctgagggtgggactcagacccaaagtgggtaaggatggaatcatccgaggtgctgagatcgcacgagttataaaagagttgatggaaggtgaggaagggaaacgaatacggagtaagatgcaggagttgaagcgtgcggcttctgctgtattgagcaaagatggatcatctactcgagctcttgaagaggttgcaaaaatttgggaaagcaaggtt

SEQ ID NO: 8 - protein, 478 aa, synthetic construct
MSESPAAPPTTAPPPHVIIVPSAGMGHLIPLAEFAKRLLPRFTFTFAVPTSGPPSSSQRDFLSSLPASIDTSFLPPVDLSDAPSDAKIETLMSLMVVRSLPSLRDLIASYVASGRRVAALVVDLFATDAIDVALELGIRPFIFFPSTAMTLSFFLHLEKLDETVSCEFAELSDPVQIPGCIPVHGKDLLDPVQDRKNDAYKWLLHHSKRYKLAEGVIVNSFEDLEGGPIRELLHPEPGKPRVYPVGPLIQAGSCEKGAAARPECLKWLDQQPRGSVLFVNFGSGGVLSTEQQNELALGLAHSQQRFLWVVRPPNDGIANATYFSVDGEIDPLKLLPEGFLEQTAGRGLVLPMWAPQIDVLSHESTGGFLTHCGWNSTLESVVHGVPLICWPLYAEQKMNCVMLTEGLRVGLRPKVGKDGIIRGAEIARVIKELMEGEEGKRIRSKMQELKRAASAVLSKDGSSTRALEEVAKIWESKV